(19) United States
(12) Plant Patent Application Publication
(10) Pub. No.: US 2011/0191925 P1
(43) Pub. Date: Aug. 4, 2011

(54) SPATHIPHYLLUM PLANT NAMED 'SWEET SEBASTIANO'

(50) ;

(76) Inventor: Petrus Olsthoorn, Honselersdijk (NL)

(21) Appl. No.: 12/658,362

(22) Filed: Feb. 4, 2010

(51) Int. Cl. A01H 5/00

(52) U.S. Cl. PLT/364

(57) ABSTRACT

A new and distinct Spathiphyllum cultivar named ‘Sweet Sebastiano’ is disclosed, characterized by larger overall plant size, durable foliage and large spathes. The new cultivar is a Spathiphyllum, typically suited for ornamental container use.

### LATIN NAME OF THE GENUS AND SPECIES

Spathiphyllum hybrid

### VARIETY DENOMINATION

‘Sweet Sebastiano’

### BACKGROUND OF THE INVENTION

The new variety originated from a planned crossing of the seed parent, an unpatented proprietary variety referred to as ‘00185-9’ with the pollen parent, the unpatented, proprietary variety referred to as ‘00057-3.’ The new variety was discovered by the inventor Petrus Olsthoorn in 2004 at a commercial greenhouse in Honselersdijk, the Netherlands.

After selecting and isolating the new cultivar, asexual reproduction of the new cultivar ‘Sweet Sebastiano’ was first performed in a commercial laboratory in Honselersdijk, the Netherlands by tissue culture in August of 2004. ‘Sweet Sebastiano’ has since produced several generations and has shown that the unique features of this cultivar are stable and reproduced true to type.

### SUMMARY OF THE INVENTION

The cultivar ‘Sweet Sebastiano’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘Sweet Sebastiano.’ These characteristics in combination distinguish ‘Sweet Sebastiano’ as a new and distinct Spathiphyllum cultivar:

1. Vigorous, tall plant.

2. Strong foliage, resistant to breakage.

3. Large spathe size.

### PARENTAL COMPARISON

Plants of the new cultivar ‘Sweet Sebastiano’ are similar to both parent varieties in most horticultural characteristics. However, ‘Sweet Sebastiano’ differs in producing larger spathes than either parent. Additionally, ‘Sweet Sebastiano’ produces larger foliage than either parent variety.

### COMMERCIAL COMPARISON

Plants of the new cultivar ‘Sweet Sebastiano’ are best compared to the commercial variety ‘Sweet Lauretta,’ U.S. Plant Pat. No. 14,004. Plants of ‘Sweet Sebastiano’ are similar to plants of ‘Sweet Lauretta’ in most horticultural characteristics, however, plants of ‘Sweet Sebastiano’ produce larger spathes than ‘Sweet Lauretta.’ Additionally, plants of ‘Sweet Sebastiano’ are taller and wider than plants of ‘Sweet Lauretta.’

### BRIEF DESCRIPTION OF THE PHOTOGRAPH

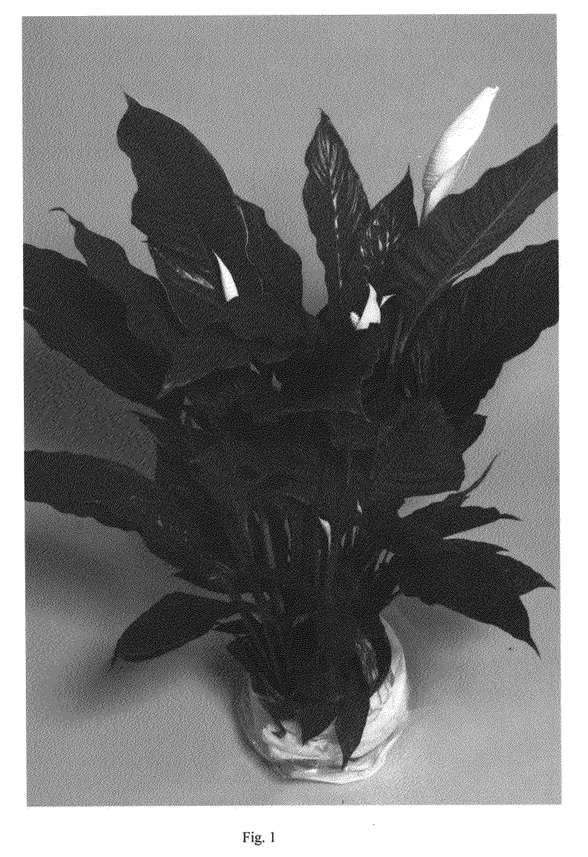

The accompanying photograph in FIG. 1 illustrates in full color a typical plant of ‘Sweet Sebastiano’ grown in a greenhouse in Honselersdijk, the Netherlands. This plant is approximately 15 months old, shown in a 24 cm pot. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart except where general terms of ordinary dictionary significance are used. The following observations and measurements describe ‘Sweet Sebastiano’ plants grown in a greenhouse in Honselersdijk, the Netherlands. Temperatures ranged from 18° C. to 20° C. at night to 19° C. to 20° C. during the day. No artificial light, photoperiodic treatments or chemical treatments were given to the plants. Measurements and numerical values represent averages of typical plant types.
Botanical classification: Spathiphyllum hybrid ‘Sweet Sebastiano’
Age of the plant described: Approximately 15 months
Container size of the plant described: 24 cm commercial container.
Propagation:
Time to initiate roots: Approximately 7-14 days from an initial tissue culture plantlet at approximately 22° C.
Time to produce rooted plantlet: Approximately 4 weeks at approximately 22° C.
Root description: Moderately fibrous, not fleshy, moderately dense, colored near RHS Yellow-White 158D.
Plant:
Growth habit: Clump-forming, upright
Plant shape: Broad obovate with flowers in between and slightly above leaf plane
Height: Approximately 98.3 cm to top of highest leaf, Approximately 105.5 cm to top of highest spathe.
Plant spread: Approximately 101.5 cm in a 24 cm
Normal pot size: 24 cm
Growth rate: High
Branching characteristics: No branching. Leaves emerge direct from base of plant.
Number of clumps of leaves: Average 7
Number of leaves per clump: Average 7
Number of leaves per plant: Approximately 49
Foliage:
Leaf:
   Arrangement.—alternate, growing directly from the soil.
   Average length (excluding petiole).—Approximately 37.2 cm.
   Average width.—Approximately 15.4 cm.
   Shape of blade.—ovare to narrow ovate.
   Aspect.—slightly curved, pointed outward to slightly upright.
   Apex.—apiculate.
   Base.—attenuate.
   Margin.—entire, moderately undulate.
   Texture of top surface.—smooth, glossy.
   Texture of bottom surface.—smooth, slightly glossy.
   Color.—Young foliage upper side: Near R.H.S. Green 143A, but darker Young foliage under side: Near in between R.H.S. Yellow-Green 144A Mature foliage upper side: Near R.H.S. Green 137B Mature foliage under side: Near R.H.S. Green 138A.
Venation:
   Type.—pinnate.
   Venation coloration upper side.—Near R.H.S. Green N137B.
   Venation coloration under side.—Near R.H.S. Yellow-Green 145B.
Petiole (excluding geniculum):
   Length.—Approximately 43.3 cm.
   Width.—At geniculum: Approximately 0.6 cm Above clump: Approximately 1.5 cm.
   Color.—Near RHS Green 137B.
   Strength.—strong.
Geniculum:
   Length.—Approximately 3.4 cm.
   Width.—Approximately 0.7 cm.
   Color.—Near RHS Yellow-Green 145A.
Inflorescence:
Arrangement: Inflorescences are carried slightly above the leaf plane
Flowering habit: Continuous
Quantity of spathes per plant: On average 4 fully open spathes in good condition at one time.
Natural flowering season: Late summer to midwinter
Time to flowering: Approximately 62 weeks
Fragrance: Moderate, sweet and pleasant (typical Spathiphyllum scent)
Self-cleaning or persistent: Persistent
Flower longevity: Flowers stay in good condition approximately 3 weeks on the plant.
Spathe:
   Aspect.—upright.
   Length.—Approximately 19.7 cm.
   Width.—Approximately 8.9 cm.
   Shape.—obovate to elliptic, strongly concave.
   Margin.—entire.
   Apex.—apiculate.
   Base.—attenuate.
   Color.—Front when opening: Near RHS White N155C, main vein and tip tinged near RHS Yellow-Green 145D Back when opening: Near RHS N155C, main vein near RHS Yellow-Green 144C, tip tinged near RHS Yellow-Green 145D Front when mature: Near RHS White N155C, main vein and tip tinged near RHS Yellow-Green 145D Back when mature: Near RHS N155C, main vein near RHS Yellow-Green 144C, tip tinged near RHS Yellow-Green 145D Fading to: not fading.
Spadix:
   Shape.—oblong.
   Tip.—obtuse.
   Base.—obtuse.
   Length.—Approximately 7.9 cm.
   Width.—Approximately 1.6 cm.
   Color.—When opening: Near in between RHS White 155D and Yellow-White 158D Mature: Near in between RHS 155D and 158D.
   Quantity of flowers per spadix.—Approximately 250.
   Spadix flower arrangement.—spirally placed on spadix.
   Spadix flower diameter.—Approximately 0.35 cm.
   Spadix flower depth.—Approximately 0.25 cm.
Peduncle:
   Length.—Approximately 73.9 cm.
   Width.—Approximately 0.5 cm.
   Color.—Near RHS Yellow-Green 145B.
   Strength.—moderately strong.
Reproductive organs:
General description of visible parts: Three stigmas are placed on top of each ovary (length of ovary approximately 0.35 cm), stigma diameter approximately 0.1 cm, colored Near RHS White N155D.
Other characteristics:
Seeds and fruits: No seeds/fruits observed.
Disease/pest resistance: Neither resistance nor susceptibility to the normal pests and diseases of Spathiphyllum has been observed.
Temperature tolerance: Indoor plant, no frost tolerance

What is claimed is:

1. A new and distinct cultivar of Spathiphyllum hybrid plant named ‘Sweet Sebastiano’ as herein illustrated and described.
